(19) United States
(12) Patent Application Publication
(10) Pub. No.: US 2023/0346283 A1
(43) Pub. Date: Nov. 2, 2023

(54) EEG HEADSET

(71) Applicant: Exsurgo Limited, Hamilton (NZ)

(72) Inventors: Faisal Almesfer, Auckland (NZ); Richard Little, Hamilton (NZ)

(21) Appl. No.: 17/916,202

(22) PCT Filed: Mar. 29, 2021
(86) PCT No.: PCT/NZ2021/050051

§ 371 Date: Sep. 30, 2022

(30) Foreign Application Priority Data
Mar. 30, 2020 (NZ) 763054

(51) Int. Cl. A61B 5/291; A61B 5/256; A61B 5/00

(52) U.S. Cl. CPC A61B 5/291; A61B 5/256; A61B 5/6803; A61B 2560/0468

(57) ABSTRACT

An EEG headset (100) comprises a base member (1) configured to be located over at least a portion of the patient's parietal and/or occipital bones in use. The base member (1) has a patient side and a non-patient side. The headset (100) further comprises a plurality of mounting members (2), wherein each mounting member (2) is rotatably connected to the base member (1) by respective rotatable connecting means (5), each rotatable connecting means (5) comprising biasing means to bias the mounting member (2) to rotate toward the patient side of the base member (1). The mounting members (5) are shaped and configured to retain the headset (100) on a patient's head, in use, and at least one of the mounting members (5) comprises an electrode engaging means (9) for engaging an electrode (14), in use. Examples of the EEG headset are substantially waterproof.

### FIELD OF INVENTION

The present invention relates to a headset for use in performing electroencephalography on a user, and in particular, but not exclusively, to an EEG headset that is useful for neurofeedback training.

### BACKGROUND TO THE INVENTION

Neurofeedback training involves a user or patient learning to manage conditions such as chronic pain (e.g. pain which persists after an injury has healed) by receiving real-time feedback on their brain activity.

During neurofeedback training, brain activity may be monitored by electroencephalography. This process requires a number of electrodes to be placed in conductive contact with the patient's scalp. Often, the electrodes will be placed in accordance with a standardised positioning system called the International 10-20 system. Electroencephalography generates an output known as an electroencephalogram (EEG).

Existing methods of obtaining an EEG may be time consuming, and may be intimidating and uncomfortable for the patient. The electrodes are often attached directly to the patient's scalp, possibly after abrasion to remove dead skin cells. If the electrodes are of a “wet” type, a conductive liquid or gel may be applied to the scalp.

In some cases, the electrodes may be connected to a net or frame to assist in locating the electrodes in the correct position.

In addition to neurofeedback training, electroencephalography may have a number of other uses including diagnosis and/or assessment of neurological conditions, for example concussion.

In some EEG methods of the prior art it may be difficult and/or time consuming to adequately prepare a set of electrodes (including any associated net or frame) which have been used with a first patient for use with a second patient.

FIGS. 1 and 2 illustrate a human skull with various bones identified, including the frontal, parietal and temporal bones.

### OBJECT OF THE INVENTION

It is an object of the invention to provide an improved EEG headset which will overcome and/or ameliorate problems with such apparatus at present.

Alternatively, it is an object of the invention to at least provide the public with a useful choice.

### SUMMARY OF THE INVENTION

An aspect of the present technology relates to an EEG headset that can be easily donned or doffed by a patient, for example using one hand.

Another aspect of the technology relates to an EEG headset that is easy to prepare for use by different patients.

Another aspect of the technology relates to an EEG headset that is substantially waterproof.

According to one aspect of the invention, there is provided an EEG headset comprising a base member configured to be located over at least a portion of the patient's parietal and/or occipital bones in use, the base member comprising a patient side and a non-patient side, the headset further comprising a plurality of mounting members, wherein each mounting member is rotatably connected to the base member by respective rotatable connecting means, each rotatable connecting means comprising biasing means to bias the mounting member to rotate toward the patient side of the base member, wherein the mounting members are shaped and configured to retain the headset on a patient's head, in use, and wherein at least one of the mounting members comprises an electrode engaging means for engaging an electrode, in use.

Preferably, each mounting member comprises at least one electrode engaging means.

Preferably, the electrode engaging means are arranged such that electrodes engaged, in use, with the electrode engaging means are arranged according to the International 10-20 system.

Preferably, the plurality of mounting members comprises a pair of lateral lower mounting members.

Preferably, the plurality of mounting members comprises a pair of upper lateral mounting members.

Preferably, the plurality of mounting members comprises a central upper mounting member.

Preferably, neither of the upper lateral mounting members is connected to any other mounting member.

Preferably the central upper mounting member is not connected to any other mounting member.

Preferably the central upper mounting member comprises a plurality of central upper mounting member portions, wherein each central upper mounting member portion is rotatably connected to an adjacent central upper mounting member portion.

Preferably, each central upper mounting portion is biased to rotate towards the patient side of the base member.

Preferably, the central upper mounting member comprises two central upper mounting member portions.

Preferably each upper lateral mounting member comprises a plurality of upper lateral mounting member portions, wherein each upper lateral mounting member portion is rotatably connected to an adjacent upper lateral mounting member portion.

Preferably, each upper lateral mounting member is biased to rotate towards the patient side of the base member.

Preferably, each upper lateral mounting member comprises three upper lateral mounting member portions.

Preferably each said mounting member portion is substantially rigid.

Preferably each rotatable connecting means comprises a flexible impermeable sleeve configured to prevent fluid ingress into the mounting member, mounting member portion and/or base member to which the rotatable connecting means is engaged.

Preferably each rotatable connecting means allows rotation about a single axis only.

Preferably each mounting member comprises chamfered longitudinal edges on a patient contacting side thereof.

Preferably each mounting member comprises a chamfered distal edge on the patient contacting side thereof.

Preferably, each lower lateral mounting member is configured to overlie at least a portion of the patient's occipital and/or temporal bones, in use.

Preferably, each lower lateral mounting member is configured to extend from the base to a position immediately posterior of the patient's ear, in use.

Preferably, each upper lateral mounting member is configured to overlie at least a portion of the patient's parietal and/or frontal bones in use.

Preferably, each upper lateral mounting member is configured to avoid overlying the patient's temporal bone, in use.

Preferably, the central upper mounting member is configured to overlie at least a portion of the patient's parietal and/or frontal bones in use.

Preferably, each mounting member portion has a patient side and a non-patient side, wherein the patient side defines a substantially concave curve.

Preferably the curve substantially conforms to the surface of the patient's head, in use.

Preferably, each mounting member comprises at least one electrode engaging means.

Preferably, the electrode engaging means are arranged such that electrodes engaged, in use, with the electrode engaging means are arranged according to the International 10-20 system.

Preferably, the headset comprises wireless and/or wired communication means.

According to another aspect of the invention, there is provided an EEG headset comprising a base member configured to sit over a patient's parietal and/or occipital bones in use, the base member comprising a patient side and a non-patient side, the headset further comprising a plurality of mounting members, wherein each mounting member is rotatably connected to the base member by a respective hinge, each hinge comprising a resilient flexible member configured to bias the mounting member to rotate toward the patient side of the base member, wherein the mounting members are shaped and configured to retain the headset on a patient's head, in use, and wherein at least one of the mounting members comprises an electrode mount to which an electrode can be mounted, in use.

According to another aspect of the invention there is provided an EEG headset comprising a base member and a plurality of mounting members configured to hold the headset in a required position on a patient's head in use, wherein each mounting member comprises at least one electrode engaging means for releasably engaging an electrode, wherein the electrode and/or the electrode engaging means comprise a seal configured to inhibit or prevent water ingress past the electrode into an interior of the mounting member.

Preferably, each electrode comprises a seal, for example an O-ring seal, which engages an inner wall of the electrode engaging means.

Preferably, at least one of the mounting members comprises a plurality of mounting member portions, each mounting member portion rotatably connected to an adjacent mounting member portion by a rotatable connecting member, wherein each rotatable connecting member comprises a flexible sealing component configured to sealingly engage the respective adjacent mounting member portion, to thereby prevent or inhibit water ingress into an interior of the mounting member portions.

According to another aspect of the invention there is provided an EEG headset comprising a base member and a plurality of mounting members configured to hold the headset in a required position on a patient's head in use, wherein each mounting member comprises at least one electrode mount to which an electrode can be mounted, in use, wherein the electrode and/or the electrode mount comprise a seal configured to prevent or inhibit water ingress past the electrode into an interior of the mounting member.

Further aspects of the invention, which should be considered in all its novel aspects, will become apparent to those skilled in the art upon reading of the following description which provides at least one example of a practical application of the invention.

### BRIEF DESCRIPTION OF THE DRAWINGS

One or more embodiments of the invention will be described below by way of example only, and without intending to be limiting, with reference to the following drawings, in which:

FIG. 1 is a perspective view of a skull with various bones identified, including frontal and parietal bones;

FIG. 2 is a side view of a skull with various bones identified, including frontal, parietal and temporal bones;

FIG. 3 is a side view of an EEG headset according to one form of the technology in position on a patient, with the patient represented diagrammatically;

FIG. 4 is a perspective view from the side and above of the EEG headset of FIG. 3 in position on a patient, with the patient represented diagrammatically;

FIG. 5 is a rear view the EEG headset of FIG. 3 in position on a patient, with the patient represented diagrammatically;

FIG. 6 is a perspective view from one side and above of an EEG headset according to another form of the technology, with connecting means and electrodes not shown;

FIG. 7 is a side view of the EEG headset of FIG. 6 with connecting means and electrodes not shown;

FIG. 8 is a front view of the EEG headset of FIG. 6 with connecting means and electrodes not shown;

FIG. 9 is a top view of the EEG headset of FIG. 6 with connecting means and electrodes not shown;

FIG. 10 is a bottom view of the EEG headset of FIG. 6 with connecting means not shown and only a single electrode shown;

FIG. 11 is a cross-section view of a mounting member portion and electrode according to one form of the technology;

FIG. 12 is an exploded view of the mounting member portion and electrode of FIG. 11;

FIG. 13 is a perspective view of an electrode according to one form of the technology;

FIG. 14 is an exploded view of the electrode of FIG. 13;

FIG. 15 is a perspective view of an electrode according to one form of the technology;

FIG. 16 is an exploded view of the electrode of FIG. 15;

FIG. 17 is a side view of an electrode according to another form of the technology;

FIG. 18 is a perspective view of the electrode of FIG. 17 seen from one end;

FIG. 19 is an exploded view of a mounting member and one form of electrode;

FIG. 20 is a cross-section view of the mounting member and electrode of FIG. 19 assembled together;

FIG. 21 is a perspective view from one side and above of a rotatable connecting means according to one form of the technology;

FIG. 22 is a perspective view from one side and above of a rotatable connecting means with a flexible sleeve according to one form of the technology;

FIG. 23 is a perspective view from one side and above of a rotatable connecting means according to another form of the technology, with a engaging portion rotated counterclockwise about a secondary axis of rotation;

FIG. 24 is a perspective view from one side and above of the rotatable connecting means of FIG. 15, with the engaging portion rotated clockwise about the secondary axis of rotation;

FIG. 25 is a perspective view from one side and above of a EEG headset according to one form of the technology;

FIG. 26 is a view from below of the headset of FIG. 25;

FIG. 27 is a view from above of the headset of FIG. 25;

FIG. 28 is a view from in front of the headset of FIG. 25;

FIG. 29 is a side view of the headset of FIG. 25 in position on a patient, with the patient represented diagrammatically;

FIG. 30 is a perspective view from one side and above of a EEG headset according to one form of the technology in position on a patient, with the patient represented diagrammatically;

FIG. 31 is a perspective view from one side and above of a rotatable connecting means according to one form of the technology;

FIG. 32 is a perspective view from one side and above of the rotatable connecting means of FIG. 31 provided with a flexible sleeve according to one form of the technology; and

FIG. 33 is a perspective view from one side and above of a rotatable connecting means according to another form of the technology.

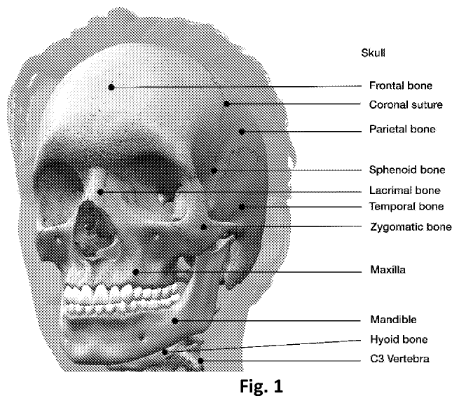

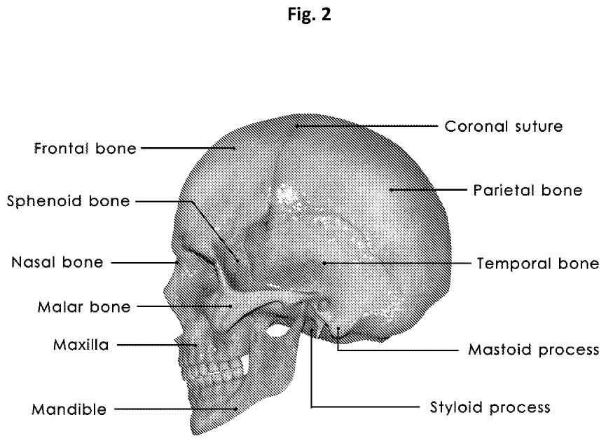

### BRIEF DESCRIPTION OF PREFERRED EMBODIMENTS OF THE INVENTION

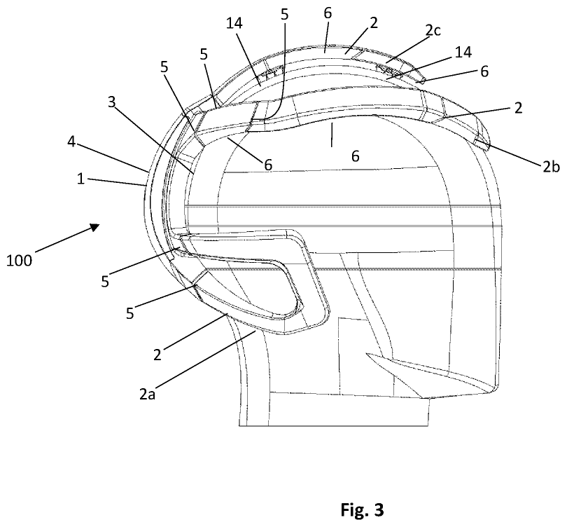

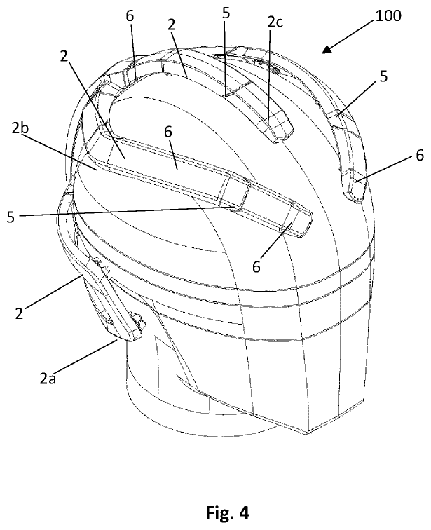

Referring first to FIGS. 3 to 5, a headset for use in performing electroencephalography on a user (hereinafter “the headset” or the “EEG headset”) is generally referenced by arrow 100. References herein to a “patient” are to any user of the EEG headset, whether or not such a user has a disease, disorder, or otherwise requires treatment.

The headset 100 comprises a base member 1 and a plurality of mounting members 2 which are rotatably attached to the base member 1. The base member 1 comprises a patient side 3 and a non-patient side 4. In some embodiments the base member 1 is shaped as an isosceles trapezoid, with the shorter of the parallel sides superior to the longer of the parallel sides when in use. At least some of the mounting members 2 may be elongate.

The mounting members 2 are rotatably attached to the base member 1 by rotatable connecting means 5, for example a hinge. The rotation of each mounting member may be such that an end of the mounting member distal the axis of rotation may move substantially radially relative to the patient's skull. In some embodiments the rotatable connections between the base member 1 and the mounting members 2 may lie on or adjacent a notional circle or ellipse, and the axes of rotation may be substantially tangential to the circle or ellipse.

The base member 1 and mounting members 2 are configured to cooperate to retain the headset 100 on a patient's head, in use. As is described further below, the mounting members 2 may have a patient side which has a complementary curve to the exterior of the patient's skull. The mounting members may rotate relative to the base member 1 to grip the patient's skull.

The rotatable connecting means 5 comprise biasing means (not shown) which bias the mounting members 2 to rotate toward the patient side 3 of the base member 1. In one embodiment the biasing means may comprise a torsion spring. Torsion springs may, for example, be connected to the base member the mounting members.

In embodiments the headset 100 comprises a pair of lateral lower mounting members 2a, a pair of upper lateral mounting members 2b, and a central upper mounting member 2c. The lower lateral mounting members 2a may be configured to extend from the base member 1 over the occipital and/or temporal bones to a position immediately posterior of the patient's ear, in use.

The upper lateral mounting members 2b may be configured to overlie the patient's parietal and/or frontal bones in use. In embodiments, the upper lateral mounting members 2b may be configured such that they do not overlie the patient's temporal bone. The upper lateral mounting members 2b may be curved when viewed from above in an inferior direction, such that the distance between the distal ends of the upper lateral mounting members 2b is smaller than the distance between the proximal ends of the upper lateral mounting members. The distance between the distal ends of the upper lateral mounting members may be between ½ and ¼, e.g. around ⅓, of a nominal width of a patient's head with which the headset is intended for use. The distal ends of the upper lateral mounting members may be at substantially the same height (that is, the same position in the superior-inferior direction) as the proximal ends, in use. The distal ends may be substantially superior to the patient's eye sockets, in use, that is, in the substantially the same position in the anterior-posterior direction as the eye sockets.

In examples the central upper mounting member 2c may be configured to overlie the patient's parietal and/or frontal bones, in use. The distal end of the central upper mounting member 2c is preferably posterior of the distal ends of the upper lateral mounting members 2b, for example, only slightly anterior of the intersection between the parietal and frontal bones.

In examples, at least some of the mounting members 2 comprise a plurality of mounting member portions 6, each of which is rotatably connected to an adjacent mounting member portion 6 by respective rotatable connecting means 5. For example, the upper mounting members 2b, 2c may comprise a plurality of mounting member portions 6. In the embodiment shown in the figures, the central upper mounting member 2c comprises two mounting member portions 6 and the upper lateral mounting members 2b each comprise three mounting member portions 6. Each mounting member portion 6 may be substantially rigid. In examples of the technology the lengths of the mounting member portions decrease along the length of each mounting member, wherein the mounting member portions adjacent the base member are longest.

Each of the rotatable connecting means 5 comprises biasing means (for example torsion springs) which bias the mounting member portions 6 to rotate towards the patient side 3 of the base member 1. In use, the mounting members 2 may rotate inwardly toward the patient side 3 of the base member 1 when the headset 100 is not mounted on a patient's head.

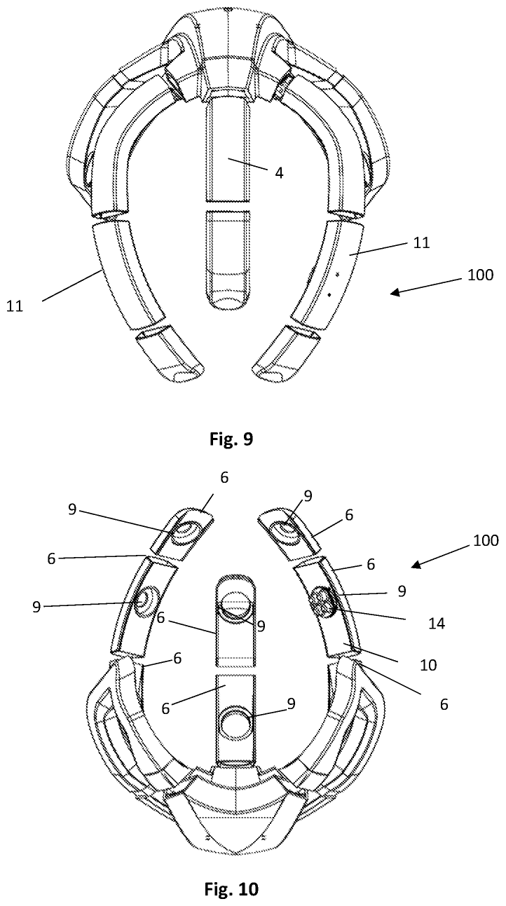

In the examples shown in FIGS. 3-10 the upper mounting members 2b, 2c are elongate and none of the upper mounting members 2b, 2c contact or are connected to any other mounting member 2 (except in as much as the mounting members 2 are connected by the base 1). In the examples shown in FIGS. 3-10, each lower lateral mounting member 2a comprises a single member which comprises a loop, for example a substantially “U” shape loop, such that the mounting member is rotatably connected to the base member 1 at two separate points. The lower lateral mounting members 2a may be configured to extend from the base member 1 over the occipital and/or temporal bones to a position immediately posterior of the patient's ear, in use. In one embodiment, an upper portion of each lower lateral mounting member may extend substantially horizontally when in use.

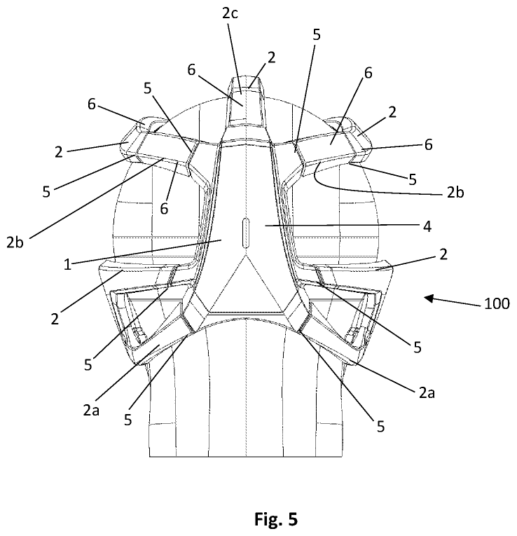

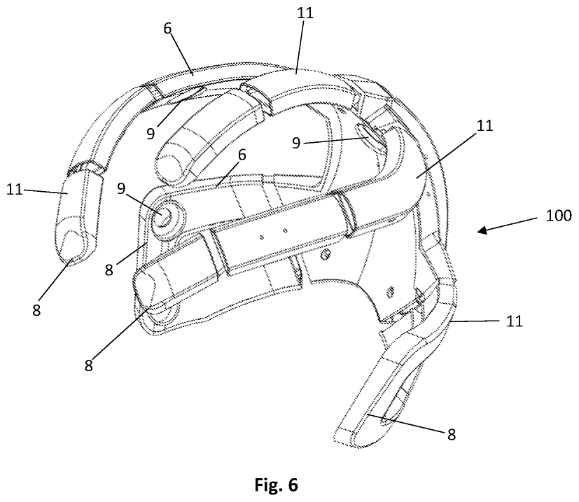

Referring next to FIGS. 6-10, in some embodiments the longitudinal edges 7 of the mounting members 2, in particular the edges of the patient contacting side, are chamfered or rounded, as best seen in FIG. 8. This may ensure the mounting members do not have sharp edges, and may assist in allowing the mounting members to move easily through the patient's hair. The edges 8 at the ends of each mounting member 2 may also be chamfered, as best seen in FIG. 6.

Referring back to FIGS. 6-10 in particular, at least one of the mounting members 2 comprises at least one electrode engaging means 9, e.g. an electrode mount. In examples, each mounting member 2 comprises an electrode engaging means 9. In further examples, each mounting member 2 comprises a plurality of electrode engaging means 9 or mounts. In examples, the base member 1 also comprises at least one electrode engaging means 9. In some embodiments the electrode engaging means 9 are arranged such that electrodes connected to the electrode engaging means 9 are arranged according to the International 10-20 system. In examples, the electrodes may be positioned at one or more, or all of, positions Fp1, Fp2, F3, Fz, F4, C3, CZ, C4 and Pz. In addition, positions A1 and A2 may be used for reference and bias. In examples, each mounting member portion 6 comprises an electrode engaging means 9 or mount.

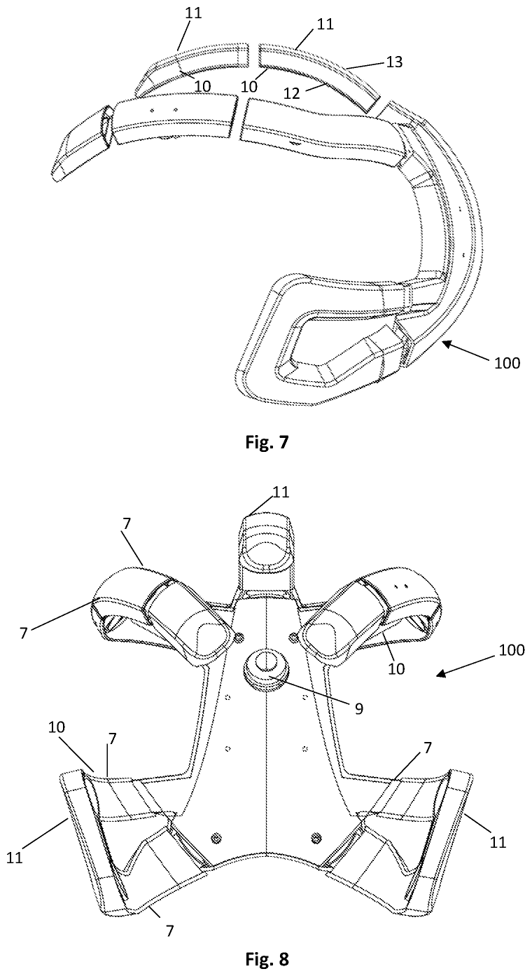

Each mounting member portion 6 may comprise a patient side 10 and a non-patient side 11. The patient side 10 of the mounting member portions 6 may comprise a substantially concave curved surface 12, as seen in FIG. 7. The curve of the concave curved surface 12 may substantially conform to the curve of the patient's head. In embodiments the non-patient side 11 of the mounting member portion 6 may have a similar convex curve 13, such that the mounting member portion 6 has a substantially constant thickness.

In preferred embodiments, each of the rotatable connections referred to above allows rotation in a single axis only, e.g, one which causes distal ends of the mounting member to move substantially radially, or, if rotation about a secondary axis is allowed, the rotation is preferably very limited (for example to around 10 degrees or less) and the secondary rotational axis is preferably substantially aligned with a longitudinal axis of the mounting member. In examples, the rotatable connections do not allow rotation about an axis which is normal to the surface of the patient's head.

This lack of lateral flexibility in the rotatable connections, in combination with the rigidity of the mounting member portions 6, results in electrodes 14 engaged with the electrode engaging means being positioned with sufficient accuracy when the headset 100 is correctly positioned on the patient's head, without the need for additional adjustment. This may mean that the headset 100 is much quicker and easier to install on the patient's head than some prior art systems which require electrodes to be positioned individually, or which may at least require adjustment of the electrode positions before use. In preferred embodiments the headset 100 can be donned and doffed by the patient using only one hand.

In examples, the headset 100 is configured to be easily disinfected and/or washed. In examples, the headset 100 may be adapted to be capable of being fully immersed in a liquid during cleaning and/or disinfecting, without the liquid penetrating an interior of the headset 100.

In examples, the electrode engaging means 9 are configured to releasably engage the electrodes 14, so that the electrodes 14 can be easily removed and replaced between uses, in particular, between uses by different patients.

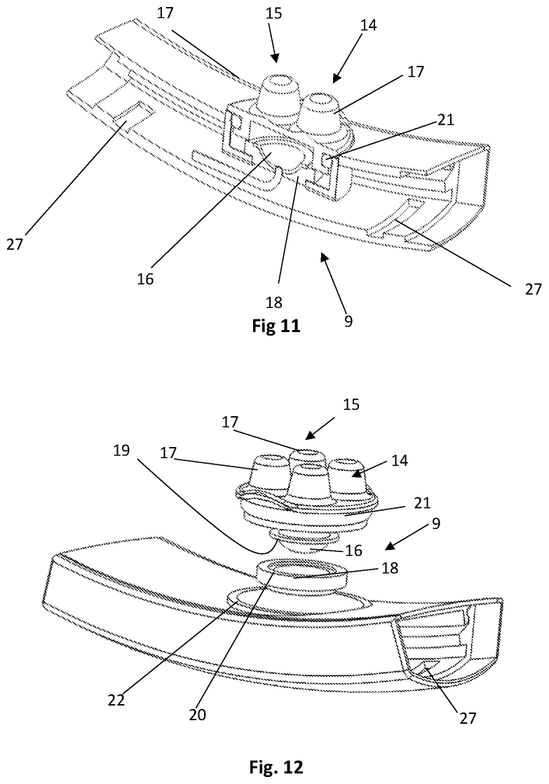

Referring next to FIGS. 11 and 12, one example of an electrode 14 is shown mounted to an electrode engaging means 9.

The electrode 14 comprises a patient contacting portion 15 and an electrical connection portion 16. The patient contacting portion comprises 15 at least one, and more preferably four conductive protrusions 17 which are configured to engage the patient's scalp when in use. In the embodiment shown the protrusions 17 are substantially frustoconical.

The electrical connection portion 16 of the electrode 14 is configured to a form an electrical connection with an electrode connection portion 18 of the electrode engaging means 9. In the embodiment shown the electrical connection portion 16 of the electrode 14 has an outer surface portion 19 which is a surface of revolution, preferably a part-spherical cap. The outer surface portion 19 engages a complementary conductive surface 20 of the electrode connection portion 18 of the electrode engaging means 9. In embodiments the electrode connection portion 18 of the electrode engaging means 9 may be biased outwardly (e.g. towards the electrode 14) to ensure that firm contact is made between the outer surface portion 19 and the conductive surface 20.

The electrode connection portion 18 of the electrode engaging means 9 is in electrical connection with a transmitting means and/or a processing means of the EEG headset. Such transmitting and/or processing means may be provided within the base member 1. In addition or alternatively, the headset may be provided with one or more ports to receive a wired connection and/or an integrated data cable. In use, the headset may transmit EEG data to a computer (for example a desktop, laptop, tablet or smartphone). In examples, the computer may operate a suitable neurofeedback program or application. In examples the data may be transmitted in real time. In other examples some or all of the data may be stored by the headset.

In embodiments a battery is also provided within the base member 1, preferably within a watertight housing.

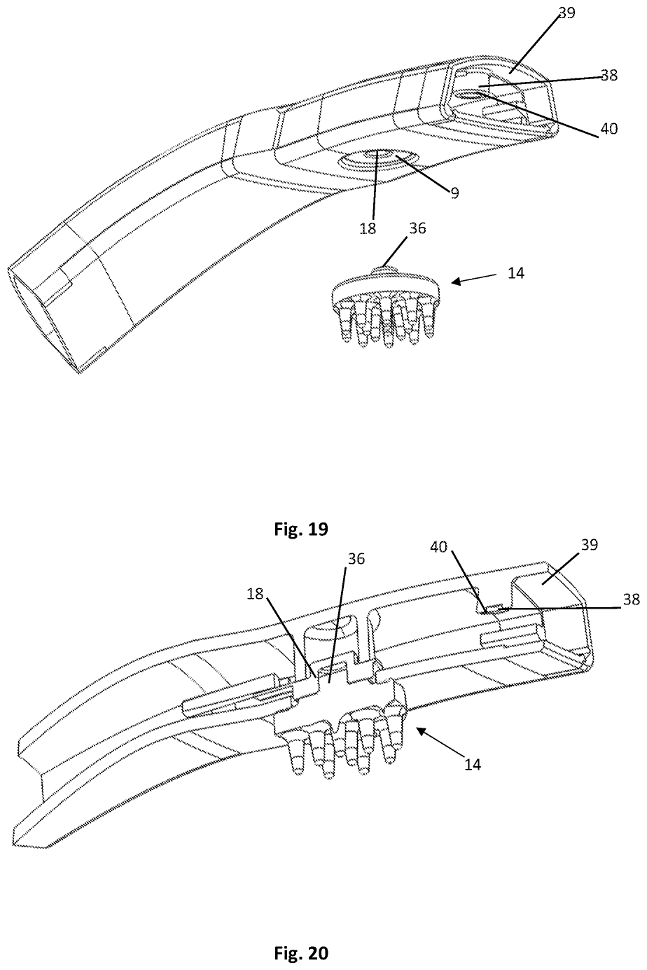

In examples the electrode 14 is provided with an O-ring seal 21 which engages a cylindrical inner wall 22 of the electrode engaging means 9 in use, to thereby prevent ingress of fluids past the electrode (e.g. between the electrode and an adjacent surface of the electrode engaging means) to an interior of the mounting member. In the embodiment shown the friction between the O-ring seal 21 and the inner wall of the electrode engaging means 9 is sufficient to hold the electrode 14 in place within the electrode engaging means 9. However, other releasable fastening means may be provided to secure the electrode 14 in position, as described further below. For example, the electrode 14 may comprise a threaded portion which engages a complementary threaded portion of the electrode engaging means 9, or the electrode 14 may engage the electrode engaging means 9 in a snap-fit engagement, as described below with reference to FIGS. 19 and 20. In both the threaded engagement and snap-fit engagement embodiments an O-ring or other seal is preferably provided to ensure no fluid enters the mounting means during cleaning or disinfecting.

In alternative embodiments (not shown) the electrode engaging means 9 may comprise a seal, for example an O-ring, which engages a suitable surface (e.g. a cylindrical outer surface) of the electrode 14.

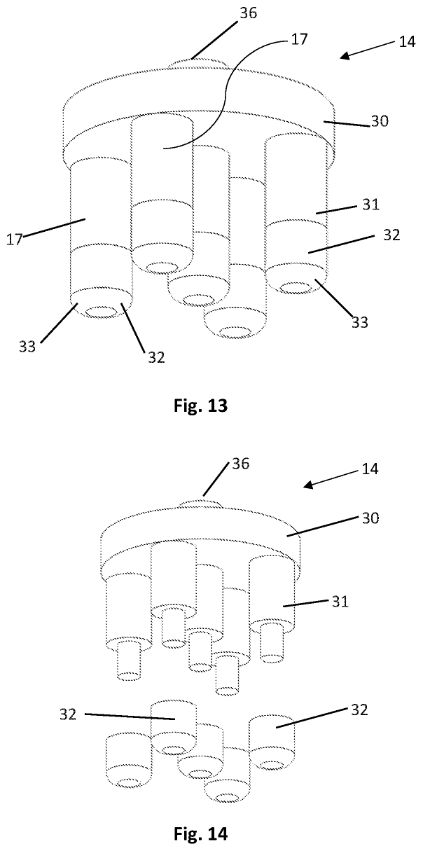

Referring next to FIGS. 13 and 14, in one example the electrode 14 may comprise a base part 30 formed from a highly conductive thermoplastic. Base portions 31 of the conductive protrusions 17 may also be formed from the thermoplastic. In examples the base part 30 and the base portions 31 may be formed integrally, for example moulded together.

Each conductive protrusion 17 may comprise a tip 32 made from a silver silicon compound. The silver silicone tips are connected to the base portions 31 of the conductive portions 17, for example by overmoulding. In examples the tips 32 have rounded ends 33.

In examples each conductive protrusion 17 has a diameter of 4 mm and a length of 10 mm. In the example shown each electrode 14 may comprise five conductive protrusions 17. Such electrodes may be particularly suitable for penetrating thick hair.

In one form the silver silicon comprises:
a conductive filler (e.g comprising approx. 10 μm Ag flakes);
silicone rubber (e.g. Ecoflex™ 00-30 1:1 mix ratio); and
a solvent (e.g. 4-methyl-2-pentanone).

In one form the thermoplastic comprises graphene polylactic acid (PLA).

In some forms of the technology the silver silicone compound may not be necessary and the entire electrode 14 may be made from graphene PLA.

In the example illustrated in FIGS. 13 and 14 each electrode 14 comprises five conductive protrusions 17, with four of the protrusions 17 arranged in two rows of two protrusions, with equal spacing between the conductive protrusions in each row and between the rows, and the fifth conductive protrusion 17 provided in the centre. The base part 30 may be substantially cylindrical or disc shaped.

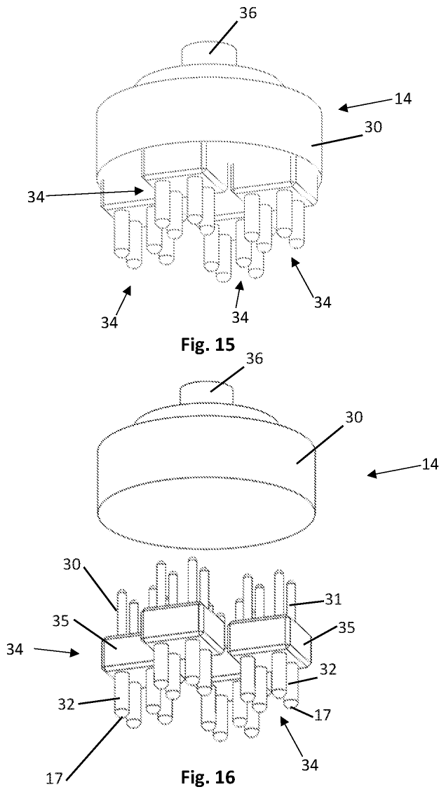

Referring next to FIGS. 15 and 16, in another form of the technology each conductive protrusion 17 comprises a tip portion 32 which is slidably (for example telescopically) mounted to a base portion 31. The conductive protrusion 17 may comprise biasing means (not shown), for example a spring, which bias the tip portion 32 away from the base portion 31, e.g. in a direction which increases the overall length of the conductive protrusion 17.

In examples the tip 32 and base 31 of the conductive protrusion 17 may be made from a copper alloy plated with a gold coating (for example 0.51 μm thick). A nickel base coat (for example 2.54 μm thick) may be used between the copper and the gold.

In one form of the technology the tips 32 are substantially 1.1 mm in diameter and substantially 5 mm long. In examples the maximum relative movement between the tip 32 and the base 31 of the conductive protrusions 17 (e.g. the “stroke”) is substantially 3 mm.

The conductive protrusions 17 may be connected to a base part 30. In examples the base part 30 may be made from a conductive thermoplastic such as the one described above. The base part 30 may be overmoulded around the bases 31 of the conductive protrusions 17.

In examples each electrode 14 comprises 16 conductive protrusions 17. The tips 32 of groups 34 of the conductive protrusions 17 may be connected together, for example by a connecting member 35, which may also be made from the thermoplastic, such that all of the tips 32 in a given group 34 move together. In the example shown in FIGS. 15 and 16 the tips 32 are grouped into four groups 34 of four conductive protrusions 17, each group 34 of tips 32 being arranged in two rows of two, with equal spacing between the conductive portions in each row, and between the rows. Grouping the tips 32 together in this way make them less susceptible to damage, and may reduce the chances of the tips 32 jamming relative to the base portions 31. This grouping also allows for comfortable pressure distribution on the scalp. The four groups 34 of conductive portions 1 may themselves be arranged in two rows of two groups.

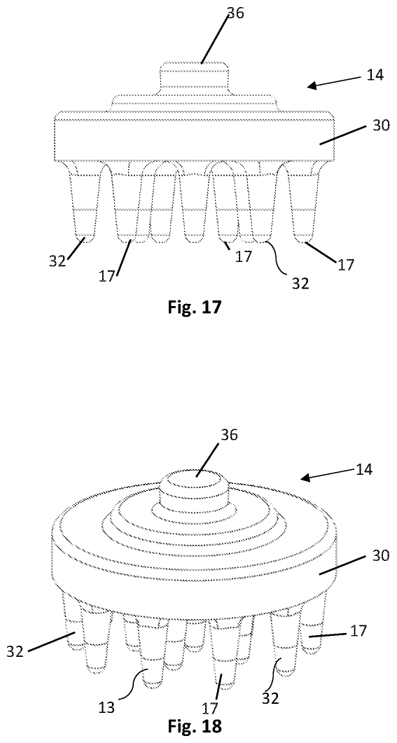

Referring next to FIGS. 17 to 20, in one form of the technology the base part 30 and conductive protrusions 17 are made from carbon nanotube silicon. In examples the tips 32 of the conductive protrusions 17 are painted or otherwise coated with a conductive coating, for example silver/silver chloride (Ag/AgCl). The Ag/AgCl coating may aid in skin-electrode conductivity by converting ionic currents in the scalp into electrical currents in the electrode, thus giving a high signal to noise ratio.

In examples the electrode 14 may comprise 16 conductive protrusions 17. The conductive protrusions 17 may be arranged to form an outer ring comprising 10 conductive protrusions 17, a concentric inner ring comprising five conductive protrusions, with a further conductive protrusion at the centre of the two rings. The conductive protrusions forming each ring may be evenly spaced apart.

Referring next to FIGS. 19 and 20 in particular, in one example the electrode 14 may comprise an engaging part 36, or example a boss, which is configured to engage the connection portion 18 of the electrode engaging means 9 in a snap fit connection. Such snap fit electrode engaging means are commercially available and are known to those skilled in the art. The connection portion 18 may be electrically connected to a transmitting means and/or a processing means of the EEG headset as described above.

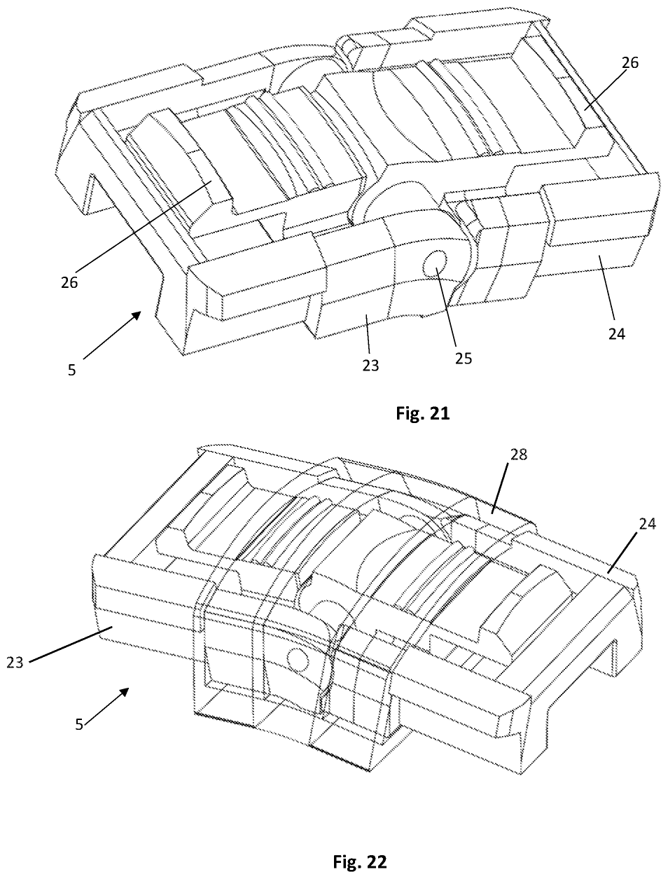

Referring next to FIGS. 21 and 22, examples of a rotatable connecting means 5 are shown. The rotatable connecting means 5 comprises a first engaging portion 23 connected to a second engaging portion 24 by a hinge formation 25. In use, each engaging portion 23, 24 engages a mounting member 2a-2c, mounting member portion 6 or the base member 1. In the embodiments shown each engaging portion 23, 24 comprises a rib 26 which engages a complementary recess 27 or protrusion in the mounting member, mounting member portion 6 or base in a snap fit. Examples of such a recess 27 are visible in FIGS. 11 and 12. In the embodiment shown each snap fit connection is releasable.

In examples the rotatable connecting means 5 comprises a flexible sleeve 28 which encircles the hinge formation 25 and at least part of the engaging portions 23, 24. The flexible sleeve 28 may be made from a substantially impermeable flexible material such as silicone or rubber. In the embodiment shown the sleeve is transparent. When the rotatable connecting means 5 is installed, an inner surface of the mounting member, mounting member portion or base member to which it is engaged engages an exterior of the flexible sleeve 28, preferably in an interference or clamping type fit. In this way the sleeve 28 prevents, or at least inhibits, ingress of water into the mounting member 2a-2c, mounting member portion 6 or base member 1 (for example during cleaning), and also prevents entanglement of the patient's hair in the hinge formation.

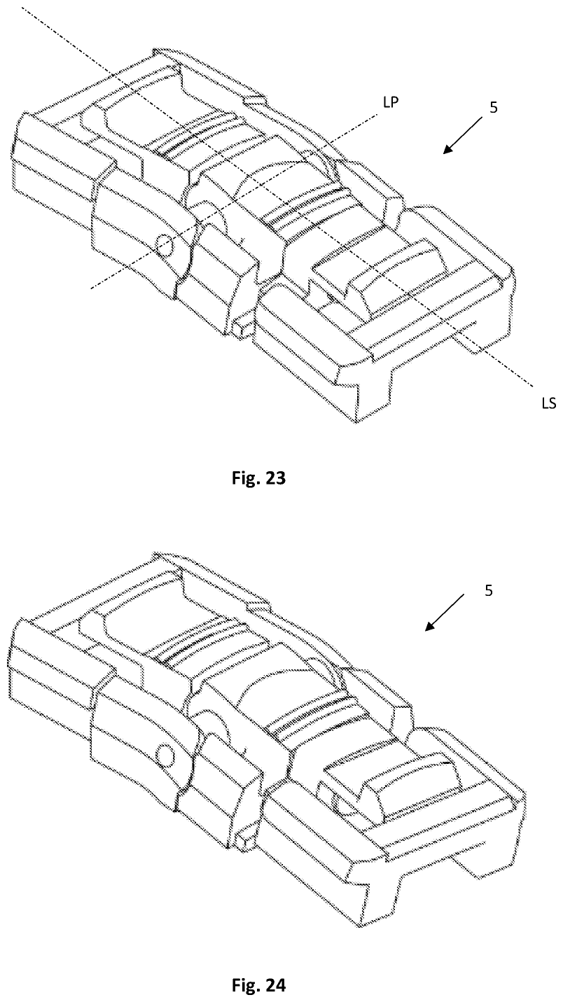

Referring next to FIGS. 23 and 24, in some examples the rotatable connecting means 5 may be configured to allow limited rotation, for example around 10°, about a secondary axis LS. The secondary rotational axis is preferably tangential to the patient's skull. In examples, the secondary axis may be substantially orthogonal to the primary axis LP of rotation.

The secondary rotational axis may be substantially aligned with a longitudinal axis of the mounting member. Allowing rotation about the secondary axis may improve the contact between the electrodes and the patient's skull.

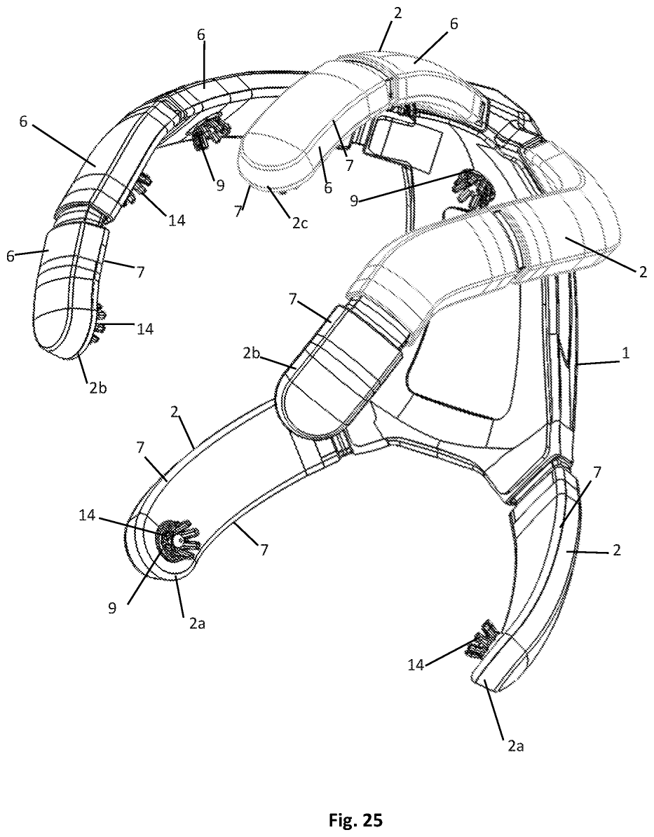

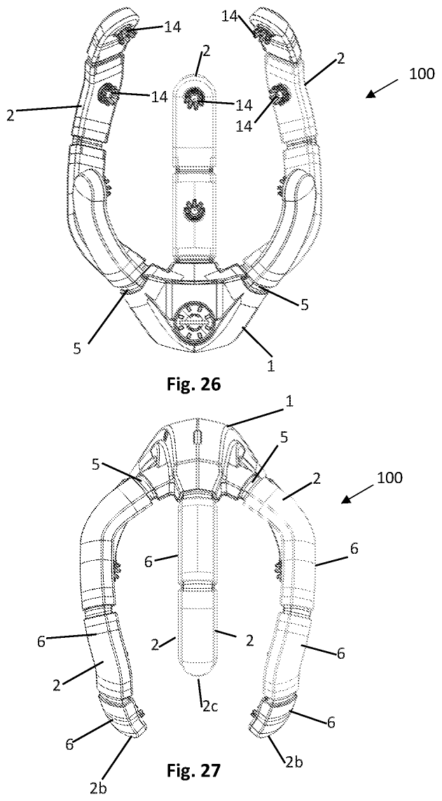

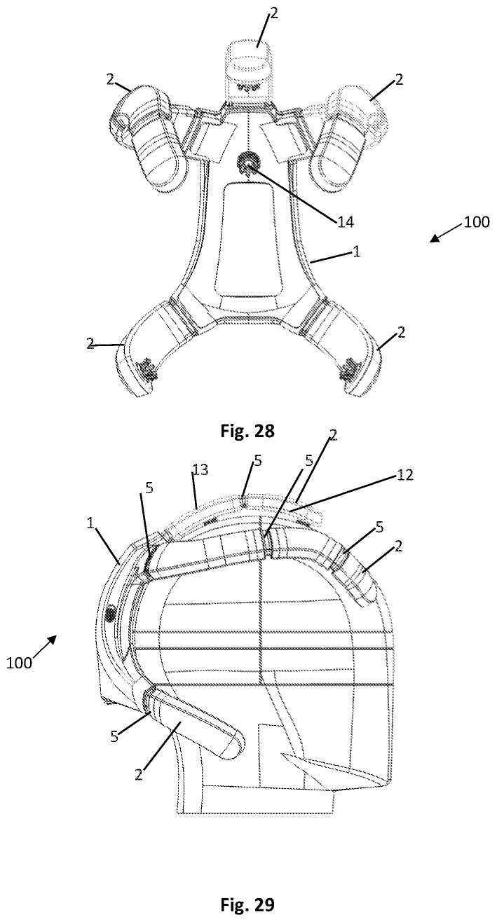

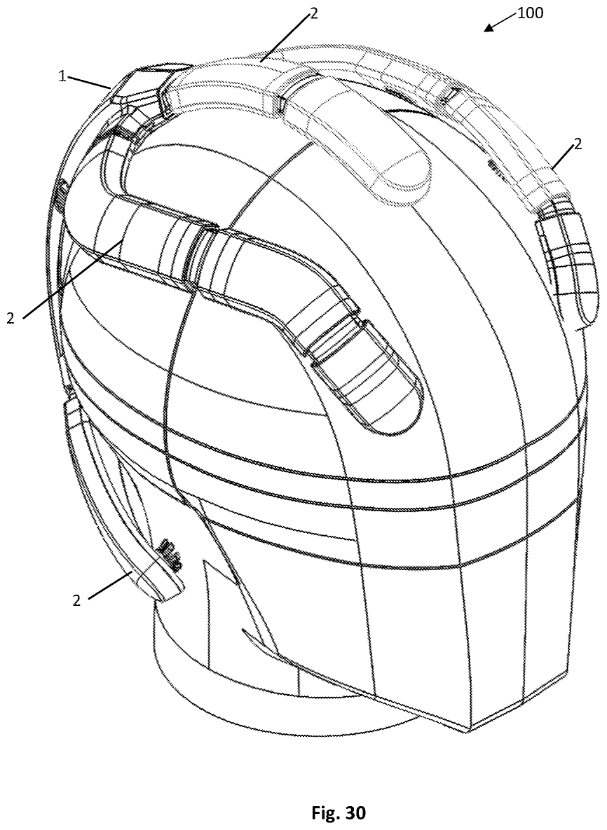

Referring next to FIGS. 25-30 another example of a headset 100 of the present technology is shown. In the example shown in these figures the upper lateral mounting members 2b are configured such that distance between the distal ends of the upper lateral mounting members is approximately equal to the distance between the proximal ends of the upper lateral mounting members.

In addition, each lower lateral mounting member 2a comprises an elongate member which is connected to the base member 1 at a single point, rather than a U shaped member connected at multiple points as shown in the example of FIGS. 3-10. The lower lateral mounting members 2a may be configured to slope downwardly from the base member 1 to a position posterior of the patient's ear, for example below the mastoid process, in use, as best seen in FIG. 29.

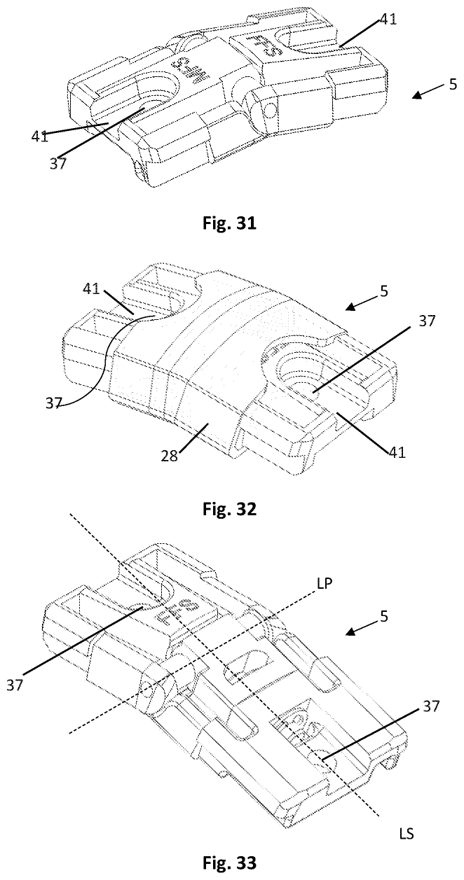

Referring next to FIGS. 31 and 32, another example of a of a rotatable connecting means 5 according to one form of the invention is shown. The rotatable connecting means 5 comprises a recess which is engaged by a projecting portion 38 of a mounting member 2a-2c, mounting member portion 6 or the base member 1, to connect the rotatable connecting means 5 to the mounting member 2a-2c, mounting member portion 6 or the base member 1. One example of such a projecting portion 38 is shown in FIGS. 19 and 20. The projecting portion 38 projects away from an internal wall 39 of the mounting member 2a-2c, mounting member portion 6 or the base member 1, but does not project externally of the mounting member.

In use at least the tip 40 of the projecting portion 38 engages the recess 37 in a snap fit in order to engage the rotatable connecting means 5 with the mounting member 2a-2c, mounting member portion 6 or the base member 1. In the example shown the recess 37 is provided at the base of a channel formation 41 in the rotatable connecting means. In examples, a recess 37 is provided at both ends of the rotatable connecting means 5.

The rotatable connecting means may be provided with a flexible sleeve 28 as described above. As shown in FIG. 33, in one embodiment the rotatable connecting means 5 may be configured to rotate about a secondary axis LS (although such rotation may be limited to around 10 degrees), as with the example shown in FIGS. 23 and 24.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

1. An EEG headset comprising a base member configured to be located over at least a portion of the patient's parietal and/or occipital bones in use, the base member comprising a patient side and a non-patient side, the headset further comprising a plurality of mounting members comprising:
a pair of lower lateral mounting members;
a pair of upper lateral mounting members; and
a central upper mounting member,
wherein each mounting member is rotatably connected to the base member by respective rotatable connecting means,
each rotatable connecting means comprising biasing means to bias the mounting member to rotate toward the patient side of the base member, wherein the mounting members are shaped and configured to retain the headset on a patient's head, in use, and wherein at least one of the mounting members comprises an electrode engaging means for engaging an electrode, in use.

2. The EEG headset of claim 1, wherein each mounting member comprises at least one electrode engaging means.

3-4. (canceled)

5. The EEG headset of claim 1, wherein each lower lateral mounting member is configured to overlie at least a portion of the patient's occipital and/or temporal bones, in use.

6-7. (canceled)

8. The EEG headset of claim 1, wherein each upper lateral mounting member comprises a plurality of upper lateral mounting member portions, wherein each upper lateral mounting member portion is rotatably connected to an adjacent upper lateral mounting member portion.

9. (canceled)

10. The EEG headset of claim 1, wherein each upper lateral mounting member comprises three upper lateral mounting member portions.

11. The EEG headset of claim 1, wherein each upper lateral mounting member is configured to overlie at least a portion of the patient's parietal and/or frontal bones in use.

12. The EEG headset of any one of claims 1, wherein each upper lateral mounting member is configured to avoid overlying the patient's temporal bone, in use.

13. The EEG headset of any one of claims 1, wherein neither of the upper lateral mounting members is connected to any other mounting member.

14. (canceled)

15. The EEG headset of claim 1, wherein the central upper mounting member is not connected to any other mounting member.

16. The EEG headset of claim 1, wherein the central upper mounting member comprises a plurality of central upper mounting member portions, wherein each central upper mounting member portion is rotatably connected to an adjacent central upper mounting member portion.

17. The EEG headset of claim 16, wherein each central upper mounting portion is biased to rotate towards a patient side of the base member.

18. The EEG headset of claim 16, wherein the central upper mounting member comprises two central upper mounting member portions.

19. The EEG headset of claim 16, wherein the central upper mounting member is configured to overlie at least a portion of the patient's parietal and/or frontal bones in use.

20. (canceled)

21. The EEG headset of claim 1, wherein each rotatable connecting means comprises a flexible impermeable sleeve configured to inhibit or prevent fluid ingress into the mounting member, mounting member portion and/or base member to which the rotatable connecting means is engaged.

22. The EEG headset of claim 1, wherein each rotatable connecting means allows rotation about a single axis only.

23. The EEG headset of claim 1, wherein each mounting member has a patient side and a non-patient side, wherein the patient side defines a substantially concave curve which substantially conforms to the surface of the patient's head, in use.

24-26. (canceled)

27. The EEG headset of claim 1, wherein each mounting member comprises at least one electrode engaging means.

28. (canceled)

29. The EEG headset of claim 1, wherein each mounting member comprises at least one electrode engaging means for releasably engaging an electrode, wherein the electrode and/or the electrode engaging means comprise a seal configured to inhibit or prevent water ingress past the electrode into an interior of the mounting member,
at least one of the mounting members rotatably connected to the base by a rotatable connecting member,
wherein the rotatable connecting member comprises a flexible sealing component configured to sealingly engage the mounting member and the base, to thereby prevent or inhibit water ingress into an interior of the mounting member.

30. (canceled)

31. The EEG headset of claim 29, wherein the seal is an O-ring seal.

32. The EEG headset of claim 29, wherein at least one of the mounting members comprises a plurality of mounting member portions, each mounting member portion rotatably connected to an adjacent mounting member portion by a rotatable connecting member, wherein each rotatable connecting member comprises a flexible sealing component configured to sealingly engage the respective adjacent mounting member portion, to thereby prevent or inhibit water ingress into an interior of the mounting member portions.

33. (canceled)
